(19) United States
(12) Patent Application Publication
(10) Pub. No.: US 2012/0150098 A1
(43) Pub. Date: Jun. 14, 2012

(54) Method and System for Mitigating Current Concentration in Electrokinetic Drug Delivery

(75) Inventors: Robert W. Etheredge, Natick, MA (US); Michael S. Barsness, Oxford, MA (US)

(73) Assignee: Nitric Biotherapeutics, Inc.

(21) Appl. No.: 13/144,660

(22) PCT Filed: Jan. 14, 2010
(86) PCT No.: PCT/US10/21083

§ 371 Date: Feb. 29, 2012

Related U.S. Application Data
(60) Provisional application No. 61/144,590, filed on Jan. 14, 2009.

(51) Int. Cl. A61M 37/00

(52) U.S. Cl. 604/20

(57) ABSTRACT

An electrokinetic apparatus to apply medicament to a treatment site of a mammalian user, the apparatus including: a segmented active electrode; a medicament matrix having one side abutting the segmented active electrode and another side adapted to contact a surface of skin over the treatment site, wherein the matrix includes at least one current direction barrier suppressing transverse current flow through the matrix.

### CROSS REFERENCE TO RELATED APPLICATIONS

The present application is a 35 U.S.C. §371 national phase application from, and claiming priority to, International Patent Application No. PCT/US2010/021083, filed on Jan. 14, 2010, which is entitled to priority under 35 U.S.C. §119(e) to U.S. Provisional Application No. 61/144,590, filed on Jan. 14, 2009, the entire disclosures of which are incorporated by reference herein as if each is being set forth herein in its entirety.

### BACKGROUND OF INVENTION

The present invention relates generally to applicators for electrokinetic mass transfer of substances to live tissue and particularly relates to an apparatus for electrokinetically delivering substances, e.g., a medicament, to a treatment site on, in or under the skin of a human patient. In particular, this application is directed to electrokinetic delivery applicators for wide areas of skin to infuse medicament into a wide area treatment site and applicator for treatment sites having high resistances, such as a toenail or fingernail or area of hard skin.

Electrokinetic delivery of medicaments applies medication topically to the skin to reach a treatment site. One type of electrokinetic delivery mechanism is iontophoresis, which is the application of an electric current to the skin to enhance the permeability of the skin and thereby deliver ionic agents, e.g., ions of salts and other drugs, to the treatment site below the surface of the skin. Electrokinetic delivery methods include iontophoretic, transdermal, transmucosal cutaneous, electroosmosis, electroporation, and electromigration, any or all of which are more generally known as electrotransport, electromolecular transport or iontophoretic methods. These techniques are collectively referred to herein as electrokinetic delivery methods.

Electrokinetic delivery methods may be problematic when applied to, for example, large areas of skin, skin with highly variable impedances, or tissues with high intrinsic impedance such as toenails. Large skin treatment areas may be associated with skin conditions such as eczema, psoriasis and acne. To deliver medicament electrokinetically to a large skin treatment site, a relatively large medicament matrix is applied to the skin. A large electrical current is generally needed to electrokinetically drive sufficient medicament from the large area matrix into the skin. The matrix consists of a uniform solid phase within which is dispersed a uniform medicament formulation. Such a matrix is limited to delivering that medicament at one rate governed by the applied current from an electrode in general contact with the entire matrix. There is a need for a medicament matrix that is more responsive to the particular needs of tissues from one site to another beneath the matrix and that is capable of delivering one or more medicaments at various rates or dosages. Further, there is a need for such a matrix able to redistribute medicament particles so that the medicament particles are more evenly distributed throughout the matrix. The present invention fulfills these needs.

### SUMMARY OF INVENTION

The invention relates to an electrokinetic apparatus for applying medicament to a treatment site of a mammalian user, the apparatus including: a segmented active electrode; a medicament matrix having one side abutting the segmented active electrode and another side adapted to contact a surface of skin over the treatment site, wherein the matrix has an associated active medicament management device or system that causes medicament to flow from regions of the matrix having an abundance of medicament to regions depleted of medicament.

The active management device or system may include electrodes to apply electric fields to redistribute the charged medicament particles such that the particles are more evenly distributed in the matrix. For example, electrodes of the active management device may arranged around a peripheral side of the matrix and a varying current applied to the electrodes. The varying electric field applied by these electrodes causes the medicament to move, e.g., diffuse, through the matrix in a direction towards or away from the electrodes.

In a preferred embodiment, the mammalian user is a human.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings. In the drawings:

FIG. 1 depicts a schematic diagram of an electrokinetic applicator including a multi-electrode active electrode, a medicament matrix and a cross-section of a cartridge housing the matrix and the active electrode.

FIG. 2 depicts the cartridge in a top down view to illustrate the multiple electrodes of the active electrode.

FIG. 3 depicts a schematic diagram of an electrokinetic applicator including a multi-electrode active electrode, a counter electrode, a medicament matrix and a cross-section of cartridge, wherein the cartridge and matrix is applied to a small area of the skin.

FIG. 4 depicts an exemplary circuit diagram of a galvanic-isolated current driver.

FIG. 5 depicts a schematic diagram of an electrokinetic device having a wide-area applicator including a multi-electrode and a medicament layer, coupled to an electronic power supply and controller.

FIG. 6 depicts the results of an example application demonstrating that a medicament matrix may have regions in which the medicament is depleted and surrounding regions which have an abundance of medicament.

FIG. 7 depicts one example placement of two diffusion plates directly opposing one another to which a varying electric field can be applied.

FIG. 8 depicts one example placement of three diffusion plates to which an electric field can be applied between, for example, alternating pairs (such as 1 and 2, 2 and 3, and 1 and 3).

FIG. 9 depicts the results of an example application demonstrating migration of dye form an area under the diffusion plate to other areas in the matrix.

### DETAILED DESCRIPTION OF THE INVENTION

The invention relates generally to an electrokinetic apparatus for applying medicament to a treatment site of a mammalian user. In one embodiment, the apparatus includes: a segmented active electrode; a medicament matrix having one side abutting the segmented active electrode and another side adapted to contact a surface of skin over the treatment site, wherein the matrix has an associated active medicament management device or system that causes medicament to flow from regions of the matrix having an abundance of medicament to regions depleted of medicament.

In some embodiments, the active management device or system may include electrodes to apply electric fields to redistribute the charged medicament particles such that the particles are more evenly distributed in the matrix. For example, electrodes of the active management device may arranged around a peripheral side of the matrix and a varying current applied to the electrodes. The varying electric field applied by these electrodes causes the medicament to move, e.g., diffuse, through the matrix in a direction towards or away from the electrodes.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

A “medicament,” as used herein, refers to any type of substance or agent that is commonly considered a drug, or a candidate for use as a drug, as well as combinations and mixtures of the above.

A “disease” is a state of health of an animal wherein the animal cannot maintain homeostasis, and wherein if the disease is not ameliorated then the animal's health continues to deteriorate. In preferred embodiment's the animal is a mammal. In more preferred embodiments, the animal is a human.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. In preferred embodiment's the animal is a mammal. In more preferred embodiments, the animal is a human.

A disease or disorder is “alleviated” if the severity of a symptom of the disease or disorder, the frequency with which such a symptom is experienced by a patient, or both, are reduced.

An “effective amount” or “therapeutically effective amount” of a medicament is that amount of medicament which is sufficient to provide a beneficial effect to the subject to which the medicament is administered.

As used herein, the term “pharmaceutically-acceptable carrier” means a chemical composition with which a medicament can be combined and which, following the combination, can be used to administer the appropriate medicament to a subject.

A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology, for the purpose of diminishing or eliminating those signs.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

### DESCRIPTION

FIG. 1 depicts a schematic diagram of an embodiment of an electrokinetic medicament applicator 10 including a portable Electrokinetic Transdermal System (ETS) control unit 12, an active electrode power connector 14, a cartridge 16 that may be releasably attached to a housing for the ETS control unit, a segmented active electrode 18 and a matrix with medicament 20.

The ETS control unit 12 can be housed in a handheld device having an actuator switch to provide a manual trigger of the application of medicament by electrokinetic delivery. The ETS control unit may comprise a power system (such as, for example, a battery), a microcontroller for monitoring certain conditions (such as, for example, whether a valid cartridge is inserted in the device) and for controlling the application of current to the active electrode, and conductive circuits connecting the power system, microcontroller, actuator switch, active electrode and counter electrode. The ETS control unit, when actuated, applies current to each of the plurality of electrode segments of the active electrode. The current applied to the electrode segments may be, for example, on the order of 660 microAmps (uA). A current path includes the power connector 14, active electrode 18, medicament matrix 20 which is applied to the skin of a patient, the patient, a counter electrode 21 that is applied to the patient, and the ETS control unit. In preferred embodiments the patient is a mammal. In more preferred embodiments, the patient is a human.

The active electrode power connector 14 may include a plurality of contact pins 22 (such as, for example, 2 or 3 or 4 or 5 or more pins), each having a current limiting device, such as a current limiting diodes (CLD) 24. The diodes are arranged in electrical series with the contact pins. A conductive bus 26 provides a common connection between each pin 22 and diode 24 arrangements and the ETS control unit. The diodes each limit the current to a respective one of the contact pins 22 to, for example, 132 uA or about one fifth the total current applied by the entire electrode segments. The diodes may be selected to limit the current to each of the contact pins to a predetermined level, such as a current level determined by the total current applied to all electrodes divided by the number of contact pins 22.

The current limiting device 24 is preferably a simple, miniature current limiting device for each of the partition and/or sub-divided segments of the active electrode. The diodes 24 are one example of a current limiting device such as, by way of non-limiting examples, the generic current limiting diodes 1N5283 through 1N5314 and the CCLM0130 manufactured by Central Semiconductor Corporation. Other electronic circuit components that limit the current to each contact pin may be suitable. There are several types of current limiting diodes (CLD), such as a current regulator diode, constant current diode, and CLDs. Current regulating diodes regulate the current flowing through them to a maximum level and if current exceeds its current regulation point, it drops its terminal voltage. A constant current diode is similar to a junction field effect transistor (JFET) whose gate terminal is shorted to source. A constant current diode can automatically limit a current through a laser driver CLD over a wide range of power supply voltages. A laser driver CLD is a type of CLD that works on the principle of a quantum process whereby light is emitted due to transition of electrons from high-level to low-level energy states. CLDs are employed to ensure that excessive current does not flow to any one of the active electrodes. The CLD is preferably arranged in the housing of the device (rather than in the cartridge), so that the CLD may be reused and is not discarded with the cartridge.

Current may be distributed equally to each segment of the active electrode 18 in proportion to the number of electrodes and/or the size of the matrix corresponding to the corresponding electrode segment. The current is distributed, for example, by the bus 26 and the current limiting device 24 with each pogo pin 22. Because of the current limiting devices 22, the current flow through each segment 36 of the active electrode 18 is preferably limited and not excessive due to a small skin area or other condition that might lead to current concentration. The maximum current density applied to the matrix by each active electrode segment is generally equal to the current applied by the pogo pin 22 applied to the electrode segment divided by the area of the electrode segment.

The pins 22 may be spring biased (such as, for example, pogo pins), such that the pins are biased downward and can be deflected upwards, as shown by a double-headed arrow in FIG. 1. The active electrode power connector 14 may be mounted in a distal end of the housing for the handled device that includes the ETS control unit 12. The pins 22 may protrude from a recess in the distal end of the housing. The recess in the housing receives a connector of the cartridge. When the cartridge is mounted in the recess, the pins 22 are biased to establish electrical connection with electrode segments of an active electrode 18 in the cartridge 16. A conductive bus 26 provides a common connection between each pin 22 and diode 24 arrangements and the ETS control unit.

The cartridge 16 may have a generally cylindrical shape with an annular plastic wall 28 that defines a cylindrical recess 30 to receive the medicament matrix 20. The recess 30 may have an open face 32 which is applied to the surface of the skin or toenail to press the medicament matrix against the skin or nail. The cartridge may alternatively be embodied as an array of cartridges that are applied to various locations on the skin or nail.

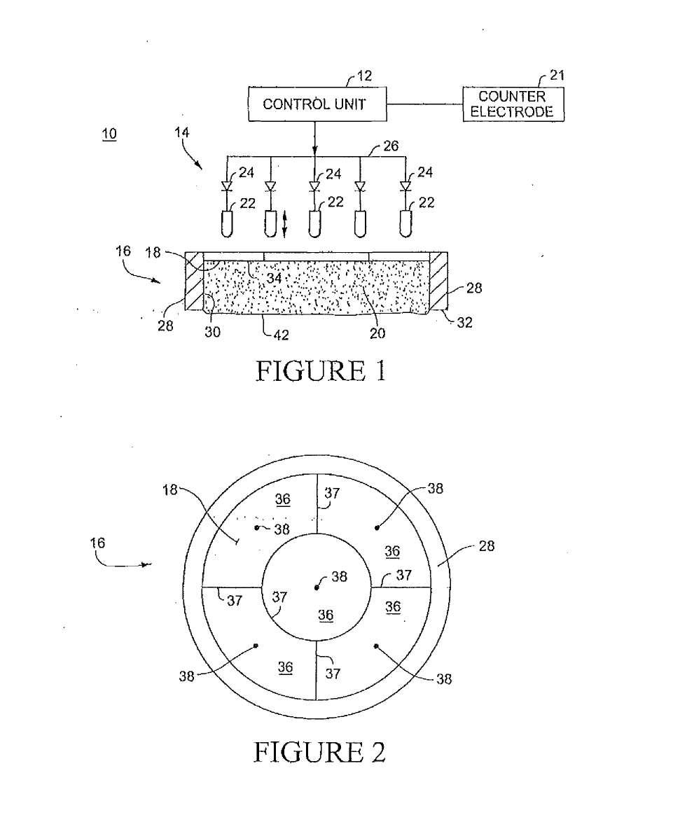

FIG. 2 shows the cartridge 16 as a cross-section. The cross section exposes a back surface of the segmented active electrode 18. The electrode 18, in one embodiment, is a thin laminated disc including metal segments 36 arranged on a substrate. The active electrode is in electrical contact with the medicament matrix 20. The substrate may be applied to the backside of the segments. Each electrode segment 36 is electrically isolated from the other active electrode segments, such as by a gap 37 between the electrode segments. The gap may be a dielectric material such as air, plastic or paper. The back surface of each electrode segment 36 includes a contact pad 38, e.g., a portion of the segment exposed through the substrate, which receives a distal end of a pogo pin. The contact pad 38 is exposed through an aperture in the cartridge to receive the pins.

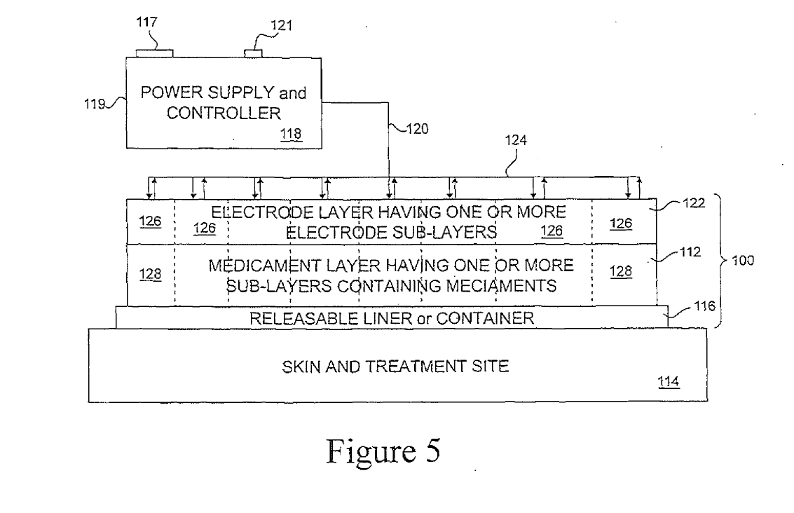

The active electrode 18 may be mounted on a surface 34 (FIG. 1) of the cartridge recess 30 that is opposite to the open face. The active electrode 18 has a first surface in contact with the medicament matrix 20 and an opposite surface abutting cartridge surface 34 and positioned to receive the pogo pins 22, when the cartridge is mounted in the device housing. The medicament matrix 20 may be a cylindrical disc mounted in the recess 30 of the cartridge. A backside of the matrix is in electrical contact with the electrode segments 36 of the active electrode 14. A front side 42 of the matrix extends slightly from the face 32 of the cartridge. The front side of the matrix is exposed and is to be placed against the skin or a nail of a toe or finger. Optionally, the front face of the matrix may include a layer of an array of microneedles 43 that are pressed into the skin to facilitate the delivery of medicament to the treatment site. A removable foil lid 116 (FIG. 5) may cover the front side of the matrix and seal the matrix in the cartridge. The lid is removed prior to the application of the matrix to the skin or nail.

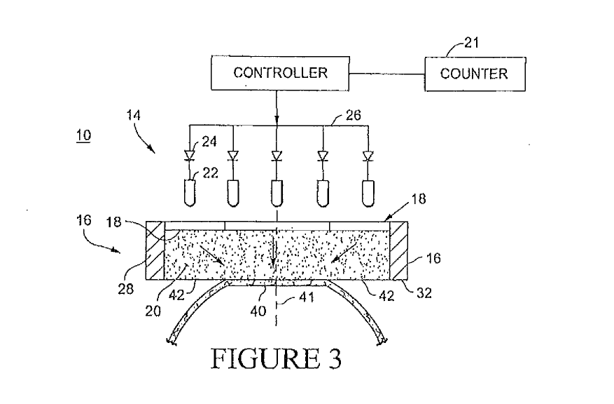

FIG. 3 is a schematic diagram of an electrokinetic applicator 10 including a multi-electrode active electrode 18, a medicament matrix 20 and a cartridge 16, wherein the cartridge and matrix is applied to a small area 40 of the skin. The small skin contact area 40 may be, for example, a cold sore which is on a curved lip. When the cartridge and matrix are applied to the small cold sore 40, only a portion of the open face 42 of the medicament matrix 20 is in contact with the skin surface. The applicator 10 is designed based on an assumption that the entire open face 42 of the matrix 20 is to be in contact with an area of the skin that is at least as large as the face 42 of the matrix. In particular, the electric current applied by the active matrix 18 is set at a level assuming that the skin contact area covers the entire area of the face 42 of the matrix.

If the skin contact area 40 is smaller than the matrix face 42, the current may become concentrated on the small skin contact area 40 as is illustrated by the arrows in FIG. 3. The limited skin contact area may receive current not only from a central active electrode segment, but also from surrounding active electrode segments which may not be axially aligned with the skin contact area. If the matrix 20 is an isotropic foam or other material that allows current to flow at an angle to the cartridge axis 41, current from active electrode segments that are not axially aligned with the skin contact area can tunnel at an angle to the axis through the matrix, to the small skin contact area 40. The electrical surface potential of each of the active electrode segments will attempt to self-adjust the voltage difference between the skin contact area and each of the active electrode segments. But for the current limiting devices 22, the self-adjustment may result in excessive voltages and/or currents to one or more of the active electrode segments. The current limiting devices 22 help prevent excessive current at any of the active electrode segments 18. However, transverse current tunneling through the matrix may funnel extra current to the small skin contact area 40 and may result in current concentration even with segmented electrodes and current limiting devices.

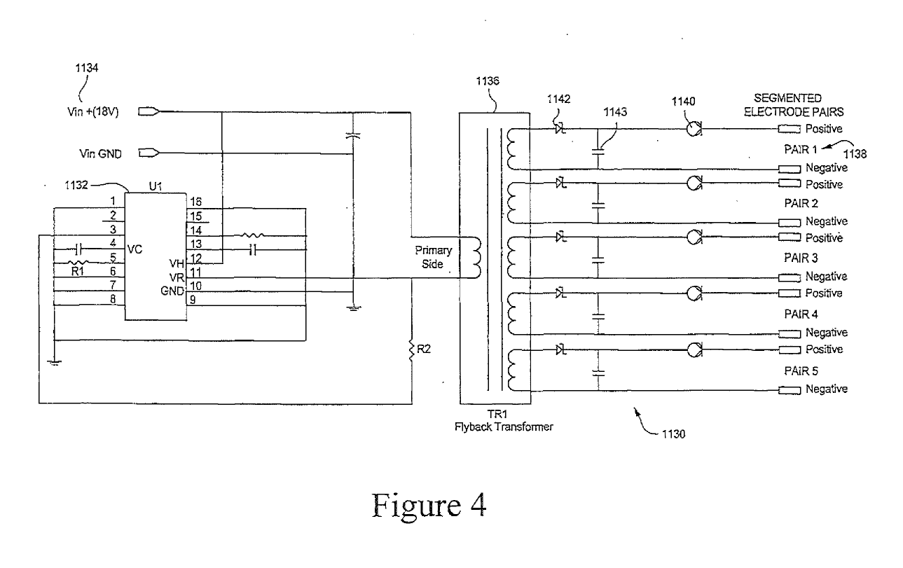

FIG. 4 is an exemplary circuit diagram of a galvanic-isolated current driver 1130. This current driver includes a galvanic isolation circuit that includes a flyback switching regulator 1136, e.g., a flyback transformer, operating in current mode topology where the secondary voltage is controlled by a microcontroller 1132 on the primary side of the flyback transformer. The flyback transformer can operate as a step-up booster transformer to generate very high voltages especially with a high secondary-to-primary turns ratio. The microcontroller 1132 regulates the current applied to the primary side of the flyback transformer. For example, the value of resistor (R2) or the ratio of resistors (R1/R2) sets the maximum voltage applied to the primary winding and hence the maximum treatment voltage applied to the secondary windings of the transformer and to the electrodes 1138. The microcontroller controls charging and shorting the primary coil to pump energy to the secondary side and into capacitors 1143 in the circuits for each of the segmented electrodes 1138. The energy pump produces a current spike that the flyback transformer 1136 steps up and generates a high voltage to be rectified 1142 into a high DC voltage. The resistors R1 and R2 may be dynamically controlled to ramp up and ramp down the voltage applied to the primary windings.

Within the loop of each pair of segmented electrodes 1138, current flows from the active (positive) to the counter (negative) electrode through a medicament matrix, the treatment site and the body of the mammalian user. The current magnitude in each current loop is limited to a value controlled by the CLD 1140 irrespective of the nail and tissue impedance. Although a high DC voltage is generated within each loop, this voltage is self regulating and it will drop entirely across the CLD 1140, nail plate and the toe. Each current loop for each electrode pair maintains a pre-set current which is galvanic-isolated in so far as the coils are isolated. Because a sufficient amount of energy is transferred to the secondary side of the flyback transformer 1136 to obtain a sufficient high DC voltage, the full current allowed by each current diode is maintained. The treatment site, such as a nail or toe, are effectively a short-circuit down stream from the current diode operating in the “limiting” mode.

FIG. 5 is a schematic view of a multi-channel iontophoretic wide-area applicator panel 100 having a medicament layer 112 formed of one or more non-woven sheets. The applicator panel may be configured, for example, as a flexible panel or pad and be incorporated in an adhesive coated pad, facemask, glove and other medicament applicator. The applicator panel is placed on the skin 114 of a patient on or over a treatment site. Before placing the applicator panel on the skin, a releasable liner 116 may be removed from a front face of the applicator panel or the applicator panel may be removed from a sealed container.

The applicator panel is connected to a power source and computer controller 118 that may be mounted on the applicator panel or attached by electrical wires 120 to an electrode layer 122 on a side of the medicament layer opposite to the skin. Electrical current through the wires 120 from the power supply and controller to an electrical distribution circuit 124 that directs current to and from individual electrodes 126. The electrical power may be delivered through separate electric current channels to each electrode 126 such that the amount of current applied to each electrode may be separately controlled by the controller 18 or other circuits associated with the distribution circuit 124.

Each electrode 126 may include an active and neutral electrical terminal. There is a neutral electrode that is unique and local to each active electrode. The current path between the active and neutral electrical terminals of each electrode passes through the medicament layer and the treatment site. Accordingly, electrical power passing through each electrode causes medicament in the medicament layer to be delivered to the treatment site as the current in the power passes between the active and neutral terminals of the electrode.

The power supply may include batteries contained in a housing with the controller or may include an adapter that plugs into a conventional electrical current supply, such as an electrical wall socket. The housing 119 for the power supply and controller portion may be releasably coupled to the applicator panel 100, wherein the connection includes the wires 120 for providing electrical power and control signals between the housing and the applicator panel. The housing may also include user interface devices, such as one or more control switches 121 and a liquid crystal (LCD) display. The one or more control switches 121 allows the user to input data and control signals into the controller, such as a medicament delivery signal or a code from a drug prescription order to indicate to the controller an amount, delivery rate and composition of medicament to be delivered to the patient. The display 117 may show to the user data generated by the controller identifying the medicament to be dispensed, application instructions, such as a location on the body to which the applicator panel is to be applied and a time period that the applicator panel is to remain on the body.

Medicament stored in the cells 128 in the medicament layer 112 of the applicator panel 100 is delivered to the skin by applying electrical current through wires and electrodes (represented by dotted lines in FIG. 12) connected to the active electrodes 126 in the electrode layer 112. The cells may each be a region of the medicament layer between an electrode and the skin. The cells may not impede the movement of medicament across the medicament layer in a direction generally perpendicular to an direction extending from the back face to the front face of the layer.

Transdermal iontophoresis is an electrokinetic process whereby charged and uncharged molecules (actives) are transferred from an electrically conductive source through the stratum-corneum and into a skin target. Mass transfer of an active across the skin is proportional to current, time, and concentration of the active in the source. Under normal circumstances a virtually infinite supply of active is available for iontophoretic delivery to the skin. Certain applications of transdermal iontophoresis use finite amounts of high-value active medicament molecules loaded into thin films of the medicament matrix. These high-value medicament molecules are preferably delivered by transdermal iontophoresis with minimal waste. The need to minimize waste is especially true for iontophoresis medicament delivery systems utilizing microneedle arrays and/or multichannel electrode designs for controlling delivery to selected skin sites. Iontophoresis applicators having multichannel electrodes and/or microneedle arrays tend to have medicament matrices, e.g., thin films, in which regions form in the matrix that have depleted of active molecules. When causing the flux rate to diminish and delivered dose to drop. In various embodiments, the invention described herein provides a means for relocating actives within the source to ensure homogeneous concentration and therefore constant delivery rate of a finite amount of active with minimal waste.

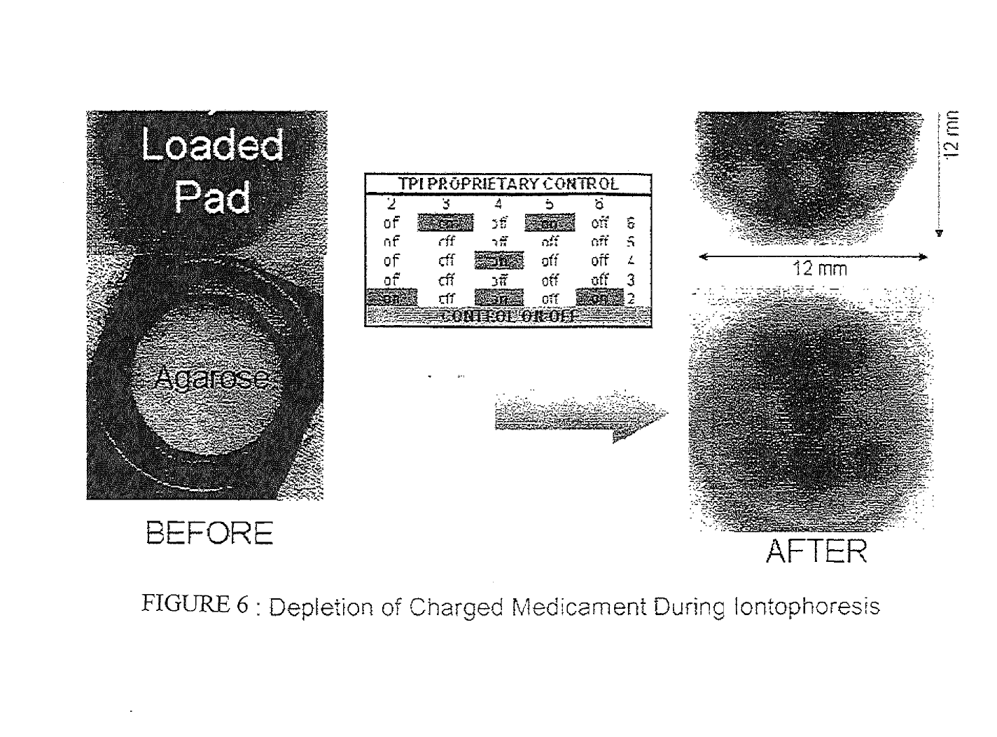

FIG. 6 depicts the results of an example application demonstrating that a medicament matrix may have regions in which the medicament is depleted and surrounding regions which have an abundance of medicament. The example medicament matrix depicted in FIG. 6 is a porous pad impregnated with a medicament that can be delivered through transdermal iontophoresis to a treatment site at or below the skin surface of a mammalian patient. A constant current was selectively applied to certain electrodes in contact with the reservoir resulting in iontophoretic delivery of dye to the underlying agarose. As can be seen in FIG. 6, areas of the reservoir directly under the active electrode were depleted over time (light colored areas in dye loaded pad) while areas under inactive electrodes were not (dark colored areas in dye loaded pad).

The medicament matrix may be included as the medicament layer 112 of a multi-channel iontophoretic wide-area applicator panel 100, as shown in FIG. 5. In one embodiment, the medicament matrix is a pad in which medicament is stored and from which medicament is delivered to a treatment site under application of current from the electrode array or through microneedles. As shown in FIG. 6 under the caption “BEFORE”, the medicament matrix may be initially uniformly loaded with medicament such that the medicament is evenly distributed across the pad. A pad of agarose was used to simulate skin and a treatment site. During the example application depicted in FIG. 6, the loaded medicament pad was applied to the pad of agarose and medicament from the pad was delivered by iontophoresis from the medicament pad into the agarose of pad.

During medicament delivery, the control system 119 may activate certain electrodes and not activate other electrodes, or the current through some electrodes may be greater than at other electrodes. One exemplary control schedule is shown in FIG. 6 for activating electrodes applied to the loaded pad for a series of medicament deliveries. As shown in the schedule, certain electrodes are on and other electrodes are off during each of the series of transdermal iontophoresis applications of medicament.

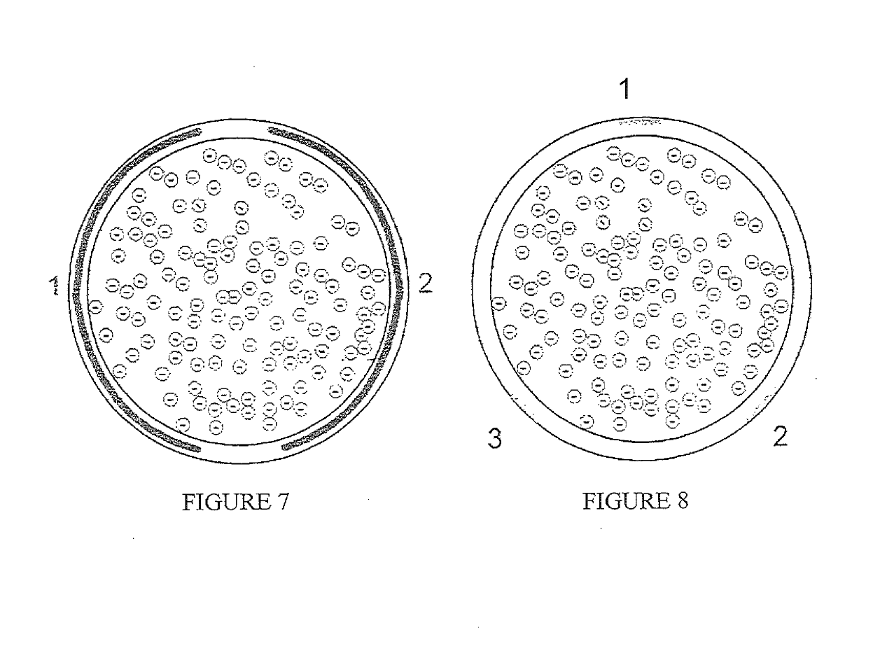

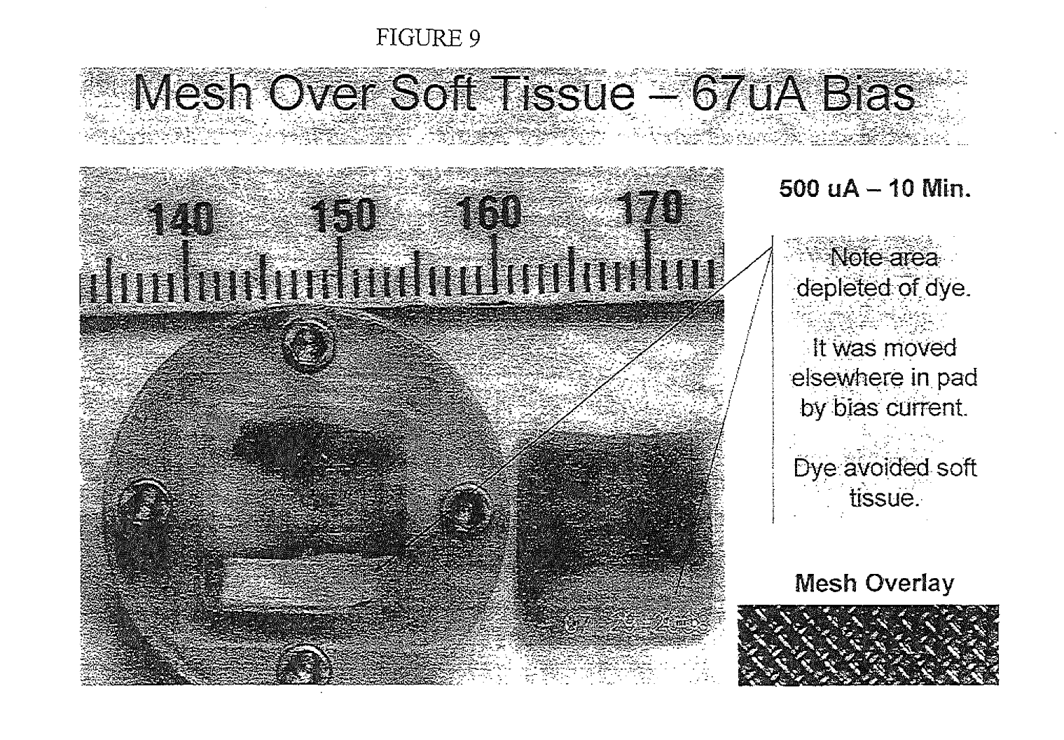

As described herein, the present invention provides a method of replenishing depleted areas of medicament in a medicament matrix during treatment. Medicament is moved from areas of high concentration to areas of low concentration by the application of an electric field in the plane of the medicament matrix. Charged medicament within the reservoir is driven in a direction away from the pole of like charge toward the pole of opposite charge. The movement of drug in the reservoir does not happen instantly and varies based upon the molecular structure of the reservoir material itself; the composition of the medicament and the direction and level of the applied field. The applied field's direction (polarity) may be rotated in order to facilitate a consistent redistribution of medicament over the entire surface by, for example, appropriately placing electrodes around the periphery of the reservoir and rotating the angel of application of the electric field. Alternatively, just two plated areas (e.g. diffusion plates) can be placed on each end of the medicament matrix and a varying electric filed can be applied. By switching the polarity back and forth, diffusion of medicament can be caused in a manner similar to the shaking of a container to effectuate diffusion of the contents therein. FIG. 7 depicts one example placement of diffusion plates directly opposing one another to which a varying electric field can be applied. FIG. 8 depicts one example placement of three diffusion plates to which an electric field can be applied between, for example, alternating pairs (such as 1 and 2, 2 and 3, and 1 and 3).

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An electrokinetic apparatus to apply medicament to a treatment site of a mammalian user, the apparatus comprising:
a first active electrode;
a second active electrode;
a medicament matrix having one side abutting the first active electrode and another side adapted to contact a surface of skin over the treatment site,
wherein the second electrode is positioned on a periphery of the medicament matrix, such that an electric field is generated by the second active electrode in the plane of the medicament matrix.

2-5. (canceled)

6. The electrokinetic apparatus of claim 1 further comprising a current limiting device electrically connected in series to each of the first and second electrodes.

7. The electrokinetic apparatus of claim 6 wherein the current limiting device is a diode.

8. The electrokinetic apparatus of claim 1 wherein each electrode is separated by a dielectric gap.

9. The electrokinetic apparatus of claim 1 further comprising a cartridge housing the matrix, first active electrode, and second active electrode, wherein the cartridge is releasably attached to a housing including a power supply and electronic control unit for the active electrodes.

10. A method of electrokinetically delivering a medicament to a treatment site of a mammalian user, the method comprising:
applying a first surface of a medicament matrix containing at least one medicament to a surface on the user;
applying a first electrode to a second surface of the medicament matrix;
generating an electrical current extending through the first electrode, medicament matrix, at least partially through the user and to a counter electrode to deliver the medicament to the treatment site;
applying a second electrode to the periphery of the medicament matrix; and
generating an electrical current in the plane of the medicament matrix to redistribute the at least one medicament within the medicament matrix.

11-16. (canceled)

17. An electrokinetic system to apply medicament to a treatment site of a mammalian user, the system comprising:
a first active electrode;
a second active electrode;
a counter electrode;
a medicament matrix having one side abutting the first active electrode and another side adapted to contact a surface of the user over the treatment site, and wherein the second active electrode is located along the periphery of the medicament matrix;
a first electrical circuit including the first active electrode and the counter electrode;
a second electrical circuit including the second active electrode, wherein the first electrical circuit is electrically isolated from the second electrical circuit;
wherein, when an electrical current is generated through the first electrical circuit, current passes from the first active electrode through the medicament matrix and at least partially through the user to the counter electrode such that the medicament is delivered to the treatment site; and
wherein, when an electrical current is generated from the second active electrode, an electric field is generated in the plane of the medicament matrix, such that medicament is redistributed within the medicament matrix.

18. The electrokinetic system of claim 17, wherein the treatment site of the user is a nail of a toe or finger of the user.

19-48. (canceled)

49. The electrokinetic apparatus of claim 1, wherein the treatment site is a nail of a toe or finger of the user.

50. The electrokinetic apparatus of claim 1, further comprising a plurality of active electrodes positioned along the periphery of the medicament matrix.

51. The electrokinetic apparatus of claim 50, further comprising a current limiting device electrically connected in series to each electrode positioned along the periphery of the medicament matrix.

52. The electrokinetic apparatus of claim 51, wherein the current limiting device is a diode.

53. The electrokinetic apparatus of claim 50, wherein each electrode positioned around the periphery is separated by a dielectric gap.

54. The method of claim 10, wherein the treatment site is a nail of the toe or finger of the user.

55. The method of claim 10, wherein the electric field generated in the plane of the matrix is varied.

56. The method of claim 10, further comprising limiting current flow to the first and second electrode via a current limiting device electrically connected in series to each electrode.

57. The method of claim 56, wherein the current limiting device is a diode.

58. The method of claim 10, further comprising applying a plurality of electrodes to the periphery of the medicament matrix.

59. The method of claim 58 wherein an alternating frequency is generated by the plurality of electrodes on the periphery of the medicament matrix.

60. The method of claim 58, wherein the electric field in the plane of the medicament matrix is varied between alternative electrode pairs.

61. The method of claim 58, further comprising limiting current flow to each electrode positioned along the periphery of the medicament matrix via a current limiting device electrically connected in series to each electrode.

62. The method of claim 61, wherein the current limiting device is a diode.

63. The system of claim 17 wherein the electric field generated by the second active electrode in the plane of the matrix varies.

64. The system of claim 17 further comprising a current limiting device electrically connected in series to each of the first and second electrodes.

65. The system of claim 64 wherein the current limiting device is a diode.

66. The system of claim 17 wherein each electrode is separated by a dielectric gap.

67. The system of claim 17 further comprising a plurality of electrodes located on the periphery of the medicament matrix; and
a plurality of electrical circuits, wherein all circuits are electrically isolated;
wherein, when an electrical current is generated from the plurality of electrodes, an electric field is generated in the plane of the medicament matrix, such that medicament is redistributed within the medicament matrix.

68. The system of claim 67, wherein an alternating frequency is generated by the plurality of electrodes.

69. The system of claim 67, wherein the electric field in the plane of the medicament matrix is varied between alternative electrode pairs.

70. The system of claim 67 further comprising a current limiting device electrically connected in series to each electrode positioned along the periphery of the medicament matrix.

71. The system of claim 70 wherein the current limiting device is a diode.

72. The system of claim 67 wherein each electrode positioned around the periphery is separated by a dielectric gap.
